(19) United States
(12) Patent Application Publication
(10) Pub. No.: US 2026/0347459 A1
(43) Pub. Date: Nov. 5, 2026

(54) A METHOD OF TREATING AUTOIMMUNE DISEASE

(71) Applicant: sThria Sciences LLC, Middletown, DE (US)

(72) Inventor: Animesh Sinha, Amherst, NY (US)

(73) Assignee: sThria Sciences LLC, Middletown, DE (US)

(21) Appl. No.: 19/483,794

(22) PCT Filed: May 15, 2024
(86) PCT No.: PCT/US2024/029399

§ 371 Date: Nov. 12, 2025

Related U.S. Application Data
(60) Provisional application No. 63/466,558, filed on May 15, 2023.

(51) Int. Cl. C07K 16/24

(52) U.S. Cl. CPC C07K 16/244; C07K 2317/76

(57) ABSTRACT

Methods are disclosed of treating an autoimmune disease in a patient comprising administering a pharmaceutical composition comprising a therapeutically effective amount of a molecular modulator that downregulates one or more IL13 genes. Methods are disclosed of treating an autoimmune disease in a patient comprising administering a pharmaceutical composition comprising a therapeutically effective amount of an antagonist to a cytokine selected from the group consisting of IL-1α, IL-1β, IL-6, TNF-α, IL-β, and IL-21 to said patient. Methods are disclosed of treating a PV patient by downregulating upregulating some of a panel of genes (protection signature genes) that are downregulated in healthy HLA-matched subjects of PV patients and by upregulating some of the protection signature genes that are upregulated in healthy HLA-matched subjects of PV patients.

### TECHNICAL FIELD

This disclosure relates to therapy for autoimmune diseases in patients.

### BACKGROUND

Autoimmune disease is characterized by the host's immune system attacking one or more self-antigens. Autoimmune diseases are a major health problem. Treatment options for autoimmune diseases are limited and largely non-specific.

### SUMMARY

In one aspect, this disclosure provides a method of treating an autoimmune disease in a patient comprising administering a pharmaceutical composition comprising a therapeutically effective amount of an IL13 antagonist to said patient. In some embodiments, the IL 13 antagonist is an antagonist to IL13α1.

In another aspect, this disclosure provides a method of treating an autoimmune disease in a patient comprising administering a pharmaceutical composition comprising a therapeutically effective amount of a molecular modulator that downregulates one or more IL13 pathway genes.

In another aspect, this disclosure provides a method of treating an autoimmune disease in a patient comprising administering a pharmaceutical composition comprising a therapeutically effective amount of an antagonist to a cytokine selected from the group consisting of IL-1α, IL-1β, IL-6, TNF-α, IL-8, and IL-21 to said patient.

In another aspect, the disclosure provides a method of treating a Pemphigus vulgaris (PV) patient by downregulating some or all of a panel of genes (protection signature genes) that are downregulated in healthy HLA-matched subjects of PV patients and/or by upregulating some or all of the protection signature genes that are upregulated in healthy HLA-matched subjects of PV patients.

In another aspect, the disclosure provides a method that first determine the protection signature genes of a subject with an auto-immune disease and then rebalance the immune system (by selectively down-regulating and/or up-regulating autoimmune relevant genes to restore “normal” healthy immune status.

Numerous other aspects are provided in accordance with these and other aspects of the invention. Other features and aspects of the present invention will become more fully apparent from the following detailed description and the appended claims.

### BRIEF DESCRIPTION OF THE DRAWINGS

FIG. 1 shows the identification of a ‘protection’ signature in HLA-matched controls (MCRs). FIG. 1A is a Venn diagram showing an overlap of 158 DEGs between the ‘control’ (534 DEGs) and ‘disease’ signatures (902 DEGs). FIG. 1B shows principle components analysis (PCA) of a subset of 155 genes out of the 158 overlapping DEGs reveals distinct spatial separation (expression level clustering) in 13 PV-A (blue), 8 PV-R (green) and 6 UMCR (purple), away from 4 MCR (red). FIG. 1C shows hierarchical clustering of ‘protection’ signature.

FIG. 2 shows IL-13Rα1 expression in monocytes. PBMCs were separated from blood of PV patients, HLA-matched (MCR) and HLA unmatched controls (UMCR) and IL-13Rα1 expression was measured by multiparameter flow cytometry in the following subsets: T helper cells (CD3+CD4+), cytotoxic T cells (CD3+CD8+), NKT cells (CD3+CD56+), NK cells (CD3−CD56+), B cells (CD19+), myeloid dendritic cells (CD11c+lin−), plasmacytoid dendritic cells (CD123+lin−), and monocytes (CD14+CD4+CD3−). No significant differences in IL-13Rα1 expression were found other than in the monocyte population (p<0.003 for UMCR vs. PV and for UMCR vs. MCR).

FIG. 3 shows Th2 cytokine expression in PV patients and healthy controls. FIG. 3A shows IL-4 expression; FIG. 3B shows IL-5 expression, and FIG. 3C shows IL-13 expression.

### DETAILED DESCRIPTION

As used herein, the word “a” or “plurality” before a noun represents one or more of the particular noun.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

“Effective amount,” “prophylactically effective amount,” or “therapeutically effective amount” refers to an amount of an agent or composition that provides a beneficial effect or favorable result to a subject, or alternatively, an amount of an agent or composition that exhibits the desired in vivo or in vitro activity. “Effective amount,” “prophylactically effective amount,” or “therapeutically effective amount” refers to an amount of an agent or composition that provides the desired biological, therapeutic, and/or prophylactic result. That result can be reduction, amelioration, palliation, lessening, delaying, and/or alleviation of one or more of the signs, symptoms, or causes of a disease, disorder or condition in a patient/subject, or any other desired alteration of a biological system. An effective amount can be administered in one or more administrations.

As used herein, a “patient” and a “subject” are interchangeable terms and may refer to a human patient/subject, a dog, a cat, a non-human primate, etc.

The terms “polypeptide,” “peptide,” and “protein” are used interchangeably and are known in the art and can mean any peptide-linked chain of amino acids, regardless of length or post-translational modification.

The term “antagonist” is known in the art. Briefly, an antagonist is a compound that binds to a target compound, such as, for example, a receptor, and prevents the target compound from functioning. An antagonist can be a chemical entity, a protein, etc. A protein antagonist can be, for example and without limitation, an antibody or its antigen-binding fragment, a fusion protein, etc.

The term “antibody” is known in the art. Briefly, it can refer to a whole antibody comprising two light chain polypeptides and two heavy chain polypeptides. Whole antibodies include different antibody isotypes including IgM, IgG, IgA, IgD, and IgE antibodies. The term “antibody” includes, for example, a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, dogs, cats, rabbits, guinea pigs, gerbils, hamsters, rats, and mice. The antibody can be a purified or a recombinant antibody.

The term “antibody fragment,” “antigen-binding fragment,” or similar terms are known in the art and can, for example, refer to a fragment of an antibody that retains the ability to bind to a target antigen. Such fragments include, e.g., a single chain antibody, a single chain Fv fragment (scFv), an Fc fragment, an Fab fragment, an Fab′ fragment, or an F(ab′)2 fragment. An scFv fragment is a single polypeptide chain that includes both the heavy and light chain variable regions of the antibody from which the scFv is derived. In addition, intrabodies, minibodies, triabodies, and diabodies are also included in the definition of antibody and are compatible for use in the methods described herein. See, e.g., Todorovska et al. (2001) J Immunol Methods 248(1):47-66; Hudson and Kortt (1999) J Immunol Methods 231(1):177-189; Poljak (1994) Structure 2(12):1121-1123; Rondon and Marasco (1997) Annual Review of Microbiology 51:257-283. An antigen-binding fragment can also include the variable region of a heavy chain polypeptide and the variable region of a light chain polypeptide. An antigen-binding fragment can thus comprise the CDRs of the light chain and heavy chain polypeptide of an antibody.

The term “antibody fragment” also can include, e.g., single domain antibodies such as camelized single domain antibodies. See, e.g., Muyldermans et al. (2001) Trends Biochem Sci 26:230-235; Nuttall et al. (2000) Curr Pharm Biotech 1:253-263; Reichmann et al. (1999) J Immunol Meth 231:25-38; PCT application publication nos. WO 94/04678 and WO 94/25591; and U.S. Pat. No. 6,005,079. The term “antibody fragment” also includes single domain antibodies comprising two VH domains with modifications such that single domain antibodies are formed.

The term “immunoglobulin” is known in the art. Briefly, the term “immunoglobulin” can refer to a polypeptide containing an amino acid sequence of at least 15 amino acids (e.g., at least 20, 30, 40, 50, 60, 70, 80, 90, or 100 amino acids, or more than 100 amino acids) of an immunoglobulin protein (e.g., a variable domain sequence, a framework sequence, or a constant domain sequence). The immunoglobulin can, for example, include at least 15 amino acids of a light chain immunoglobulin, e.g., at least 15 amino acids of a heavy chain immunoglobulin, such as a CDRH3. The immunoglobulin may be an isolated antibody (e.g., an IgG, IgE, IgD, IgA, or IgM). The immunoglobulin may be a subclass of IgG (e.g., IgG1, IgG2, IgG3, or IgG4). The immunoglobulin can be an antibody fragment, e.g., a Fab fragment, a F(ab′)2 fragment, or an scFv. The immunoglobulin can also be a bi-specific antibody or a tri-specific antibody, or a dimer, trimer, or multimer antibody, or a diabody, a DVD-Ig, a CODV-Ig, an Affibody®, or a Nanobody®. The immunoglobulin can also be an engineered protein containing at least one immunoglobulin domain (e.g., a fusion protein). Examples of immunoglobulins are known in the art.

The term “fusion protein” is known in the art. Briefly, fusion protein is a protein having two proteins or two protein domains joined together. A fusion protein can be, for example, an antibody fragment fused to the Fc fragment of another immunoglobulin. Methods of constructing fusion proteins, such as by recombinant DNA technology, are well known in the art.

The terms “self-antigen” and “auto-antigen” are used interchangeably herein.

All ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “5 to 10” or “5-10” should be considered to include the end points 5 and 10.

It is further to be understood that the feature or features of one embodiment may generally be applied to other embodiments, even though not specifically described or illustrated in such other embodiments, unless expressly prohibited by this disclosure or the nature of the relevant embodiments. Likewise, compositions and methods described herein can include any combination of features and/or steps described herein not inconsistent with the objectives of the present disclosure. Numerous modifications and/or adaptations of the compositions and methods described herein will be readily apparent to those skilled in the art without departing from the present subject matter.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Autoimmune Diseases

An autoimmune disease, characterized by the host's immune system attacking one or more self-antigens, is a major healthcare burden. There are over 100 known human autoimmune diseases, affecting between 5-10% of the population. Autoimmune diseases are the 2^{nd} or 3^{rd} leading cause of morbidity and mortality and costing the US healthcare system over $100 billion annually. Lupus, pemphigoid, myasthenia gravis, multiple sclerosis, type 1 diabetes, and pemphigus vulgaris are examples of autoimmune diseases.

Treatment options for autoimmune diseases are limited and largely non-specific or symptom oriented. No true cures are available and there is no consensus treatment guidelines. Targeted, individualized therapies are lacking.

Pemphigus vulgaris (PV) is a potentially life-threatening autoimmune blistering skin disease characterized by intraepithelial (suprabasalar) acantholysis, which is a loss of cell-cell adhesion. PV is Th2 driven and involve IgG4 autoantibodies. The primary autoantibody targets (autoantigens), Desmoglein (Dsg)-3 and Desmoglein-1, have been identified.

PV is a humorally based autoimmune disease in which the Th2 pathway is critical to promote the development of anti-Dsg 3 and anti-Dsg 1 autoantibodies. Numerous studies have established an association between specific HLA class II alleles, DRB1*0402 and DQB1*0503, and increased risk for PV. While up to 95% of PV patients carry one or both of these HLA class II alleles, the vast majority of individuals carrying 0402/0503 do not develop disease; additional susceptibility factors are required to develop disease or must be lacking to not develop disease.

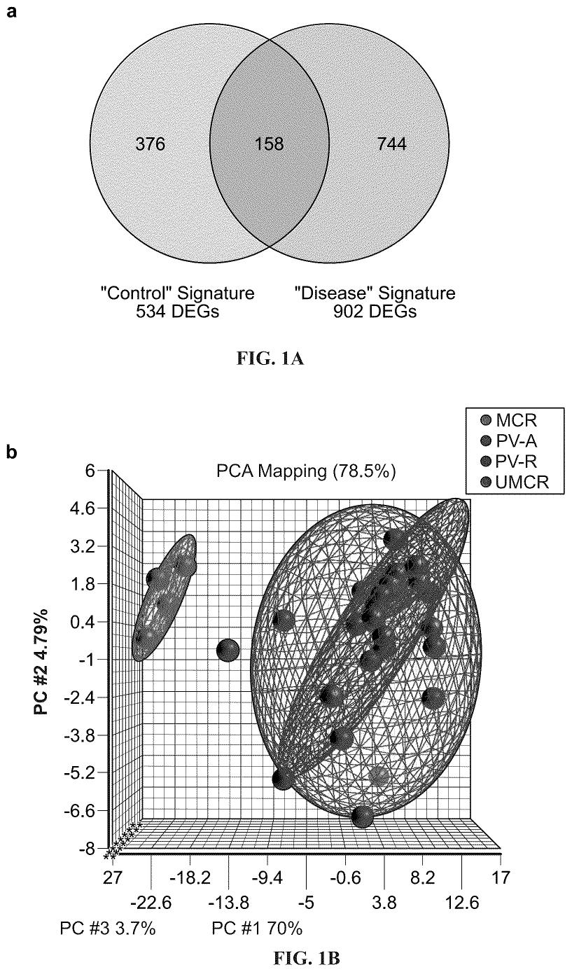

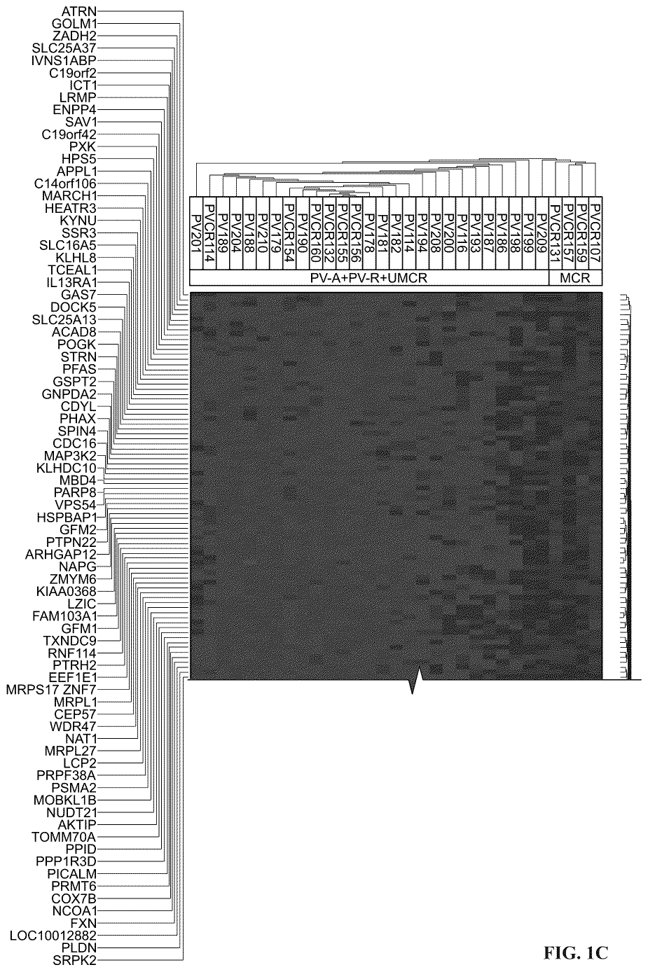

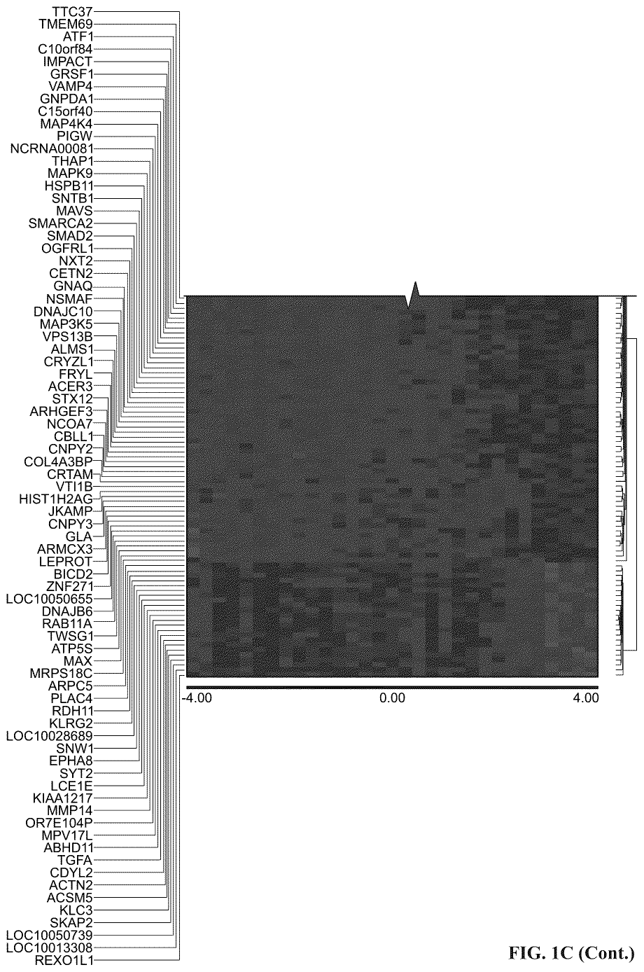

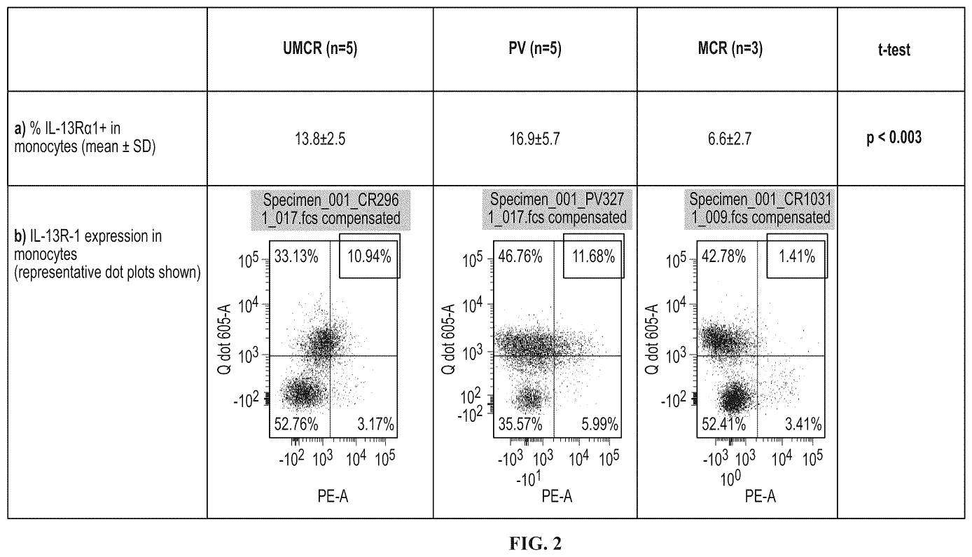

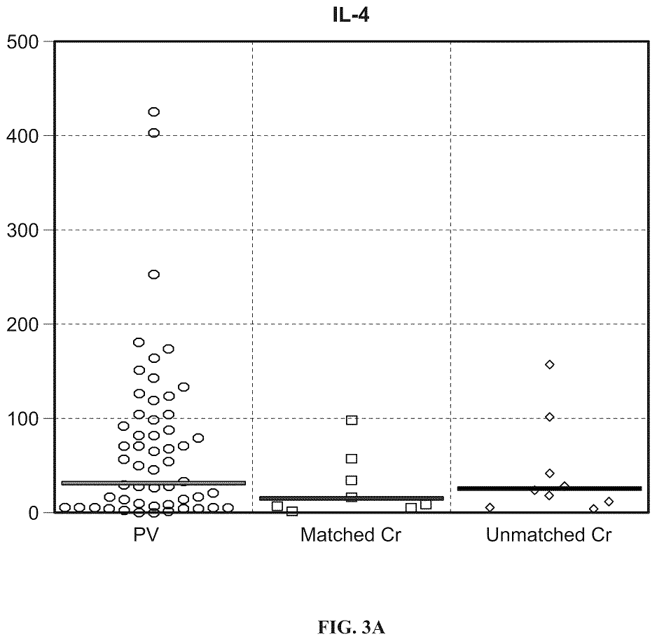

Genome-wide transcriptional analysis data have identified a group of genes that are specifically downregulated (some are upregulated) in HLA-matched controls (MCR) of PV patients, but not in HLA-unmatched controls (UMCR) of PV patients and in PV patients. Apparently, suppression of critical pathways in genetically susceptible individuals could function to protect from developing the autoimmune phenotype. See Dey-Rao R, Seiffert-Sinha K, & Sinha A A (2013) Genes and immunity 14(8):487-499; and Saha P & Geissmann F (2011) Immunology and cell biology 89(1):2-4. These genes, termed the “protection signature” genes, must be down-regulated to prevent autoimmunity in genetically susceptible individuals, who have already activated several autoimmunity related pathways. FIG. 1A, FIG. 1B, FIG. 1C, and FIG. 2.

One protection signature gene is the IL-13 receptor α1 (IL-13Rα1). As shown by genome-wide transcriptional analysis, IL-13Rα1 is downregulated in MCR control subjects but not in PV patients and UMCR subjects; and also downregulated in remittent PV patients but not in active PV patients. The IL-13Rα1 gene is downregulated in peripheral blood of MCR subjects, but not in peripheral blood of active PV patients and unmatched control subjects, as confirmed by qPCR. IL-13Rα1 is down-regulated at the protein level in peripheral blood mononuclear cells (PBMCs) of MCR, and not in peripheral blood mononuclear cells of active PV patients and unmatched control subjects, as confirmed by flow cytometry. The downregulation of the IL-13Rα1 proteins localizes to the monocytes population, but not to B cells and T cells. Monocytes form a continuum/constellation of cells (monocytes/macrophages/dendritic cells) that play key roles in antigen presentation and the initiation of normal immune, and presumably, aberrant, autoimmune responses.

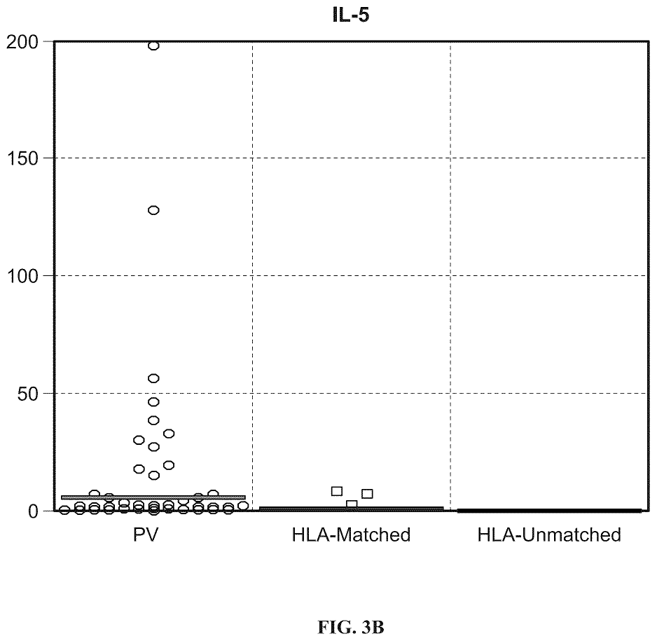

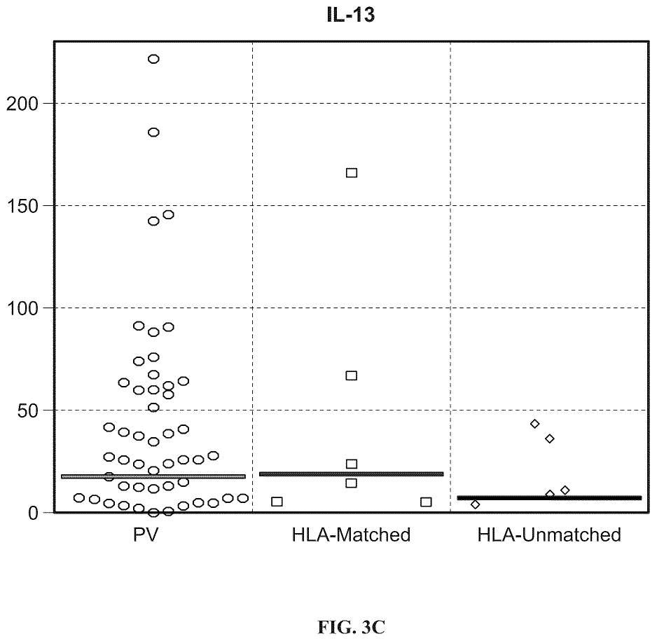

Serum cytokine expression has been analyzed in a comprehensive set of cytokines and chemokines covering several functional categories by multiplexed cytokine bead array assay (Luminex platform) using samples from 117 PV patients, 15 HLA-matched and 15 HLA-unmatched controls. FIG. 3A, FIG. 3B, and FIG. 3C. serum cytokine expression in a comprehensive set of cytokines and chemokines covering several functional categories by multiplexed cytokine bead array assay (Luminex platform) using samples from 117 PV patients, 15 HLA-matched (MCR) and 15 HLA-unmatched controls (UMCR). The Th2 cytokine IL-4 did not reveal any significant differences between the groups. The Th2 cytokine IL-5 was significantly increased in PV patients vs. both matched and unmatched control. The Th2 cytokine IL-13 in HLA-matched controls was observed at levels comparable to PV patients, but IL-13 was significantly lower in HLA-unmatched controls. Matched controls exhibit an immunological state similar to PV patients, but distinct from HLA-unmatched controls for additional cytokines such as IL-1α, IL-1β, IL-6, TNF-α, IL-8, and IL-13. These data suggest there is a state of limited immune activation in healthy controls that carry PV-HLA-susceptibility alleles.

The IL-13Rα1 gene encodes one component of the common IL-4/IL-13 receptor. Both IL-4 and IL-13 promote differentiation of Th2 cells and are essential for B cell activation and autoantibody production.

IL13 increases the expression of transcription factor STAT6 and HLA class II (known to be linked to STAT6 upregulation) in monocytes. The upregulation of HLA II in monocytes correlates with a more activated monocyte phenotype, and may result in increased (auto)antigen presentation that helps to perpetuate the autoimmune response. IL13, a key Th₂ cytokine, has been shown to be upregulated in the serum of active PV patients and MCR, but not in the serum of UMCR.

It appears that autoimmunity is not only driven by certain genes and pathways, but also the inability of patients to counteract autoimmune driving pathways—PV patients lack this ability, but MCR have this counterregulatory capacity. UMCR, not carrying the autoimmune risk alleles, have no need to downregulate this set of protection signature genes, since they do not exhibit dysregulation in disease relevant pathways.

While there is at least a partial autoimmune activation in HLA-matched controls similar to that seen in PV patients, they do not progress to disease because: they lack the additional HLA-independent factors that are required for the development of disease in patients; and they are able to “protect” from progression to disease by the ability to down-regulate disease relevant genes/pathways.

While HLA predisposes to development of PV, additional factors are at play and disease develops in patients due to both: processes independent of HLA-status that are absent in healthy controls; and a failure to suppress autoimmune activation in patients, that is achieved in genetically susceptible healthy controls.

The downregulation of IL-13Rα1 on peripheral blood monocytes can be instrumental in the protection against disease development in genetically susceptible, but healthy individuals, and also can play a role in reverting PV patients into remission.

### Molecules, Compositions and Methods

In one aspect, this disclosure provides a method of treating an autoimmune disease in a patient comprising administering a pharmaceutical composition comprising a therapeutically effective amount of an IL13 antagonist to said patient. In some embodiments, the IL13 antagonist is an antagonist to IL13α1. In some embodiments, the antagonist is an antibody, or an antigen binding fragment thereof. In some embodiments, the autoimmune disease is a Th2 T-cell mediated autoimmune disease. In some embodiments, the autoimmune disease is Pemphigus vulgaris (PV).

Immune responses driven preferentially by activated T-cell subsets are involved in the development of pathologic immune disorders, such as autoimmune diseases. Th₂ T-cell mediated autoimmune diseases involve Th₂ T-cells, but not necessarily exclusively. Th₂ cells generally mediate humoral immunity and Th₁ cells generally promote cellular immunity.

In another aspect, this disclosure provides a method of treating an autoimmune disease in a patient comprising administering a pharmaceutical composition comprising a therapeutically effective amount of a molecular modulator that downregulates one or more IL13 gene. In some embodiments, the one or more IL13 genes comprise IL13α1. In some embodiments, the autoimmune disease is a Th₂ T-cell mediated autoimmune disease. In some embodiments, the autoimmune disease is Pemphigus vulgaris (PV). The molecular modulator is one that can downregulate one or more IL13 genes. The molecular modulator can be one entity, several entities, or a system of entities. In some embodiments, the molecular modulator is an RNAi. In some embodiments, the molecular modulator is mediated by CRISPR gene editing technology, Talen technology, or zinc finger linked nuclease technology.

In yet another aspect, this disclosure provides a method of treating an autoimmune disease in a patient comprising administering a pharmaceutical composition comprising a therapeutically effective amount of an antagonist to a cytokine selected from the group consisting of IL-1α, IL-1β, IL-6, TNF-α, IL-8, and IL-21 to said patient. In some embodiments, the antagonist is an antibody. In some embodiments, the autoimmune disease is Pemphigus vulgaris (PV).

In another aspect, this disclosure discloses a method to downregulate or upregulate most, if not all, of the protection signature genes. While most of the protection signature genes are downregulated in healthy subjects carrying the same HLA susceptibility alleles as PV patients compared to PV patients, some genes within the protection signature are upregulated. Thus, the disclosed method should upregulate those protection signature genes that are upregulated in healthy subjects and downregulate those protection signature genes that are downregulated in healthy subject. PV patients cannot downregulate (or in some cases upregulate) protection signature genes, as do the healthy HLA matched controls, and therefore cannot “protect” themselves from the drivers of autoimmunity. Rebalancing the levels of the protection signature genes in patients to the levels/pattern seen the HLA matched healthy controls would then allow patients to “protect” themselves from autoimmune insult.) Thus, the disclosure provides a method of treating a PV patient by downregulating upregulating some of a panel of genes (protection signature genes) that are downregulated in healthy HLA-matched subjects of PV patients and by upregulating some of the protection signature genes that are upregulated in healthy HLA-matched subjects of PV patients. In some embodiments, the genes are downregulated or upregulated by use of siRNA, CRISPR, autoantibody blocking, molecular antagonists/agonists small molecule blocking/boosting.

In another aspect, the disclosure provides a method that first determine the protection signature genes of a subject with an auto-immune disease (any auto-immune disease) and then rebalance the immune system (by selectively down-regulating and/or up-regulating autoimmune relevant genes to restore “normal” healthy immune status. Thus, the disclosure provides a method of treating an auto-immune disease comprising: determining protection signature genes of a subject with an auto-immune disease, genes that are upregulated or downregulated in HLA-matched healthy subjects; rebalancing the immune system by selectively down-regulating genes that are downregulated in healthy HLA-matched subjects of the subject with the auto-immune disease and by upregulating some of the genes that are upregulated in healthy HLA-matched subject of the subject with the auto-immune disease. In some embodiments, the genes are downregulated or upregulated by use of siRNA, CRISPR, autoantibody blocking, molecular antagonists/agonists small molecule blocking/boosting.

### Formulating and Administering Compositions

The composition for use in the disclosed methods may be administered to a subject in need thereof by any suitable mode of administration, any suitable frequency, and at any suitable, effective dosage.

The composition for use in a disclosed method may be in any suitable form and may be formulated for any suitable means of delivery.

In some embodiments, the disclosed composition is provided in a form suitable for injection, such as subcutaneous, intramuscular, intravenous, intraperitoneal, or any other route of injection. In some embodiments, compositions for injection are provided in sterile and/or non-pyrogenic form and may contain preservatives and/or other suitable excipients, such as sucrose, sodium phosphate dibasic heptahydrate or other suitable buffer, a pH-adjusting agent such as hydrochloric acid or sodium hydroxide, and polysorbate 80 or other suitable detergent.

When provided in solution form, in some embodiments, the composition for use in a disclosed method is provided in a glass or plastic bottle, vial or ampoule, any of which may be suitable for either single or multiple use. The bottle, vial or ampoule containing the disclosed composition may be provided in kit form together with one or more needles of suitable gauge and/or one or more syringes, all of which preferably are sterile. Thus, in certain embodiments, a kit is provided comprising a liquid solution as described above, which is packaged in a suitable glass or plastic bottle, vial or ampoule and may further comprising one or more needles and/or one or more syringes. The kit may further comprise instruction for use.

The composition for use in a disclosed method can be produced by methods employed in accordance with general practice in the pharmaceutical industry, such as, for example, the methods illustrated in Remington: The Science and Practice of Pharmacy (Pharmaceutical Press; 21st revised ed. (2011) (hereinafter “Remington”).

In some embodiments, the composition for use in a disclosed method comprise at least one pharmaceutically acceptable vehicle or excipient. These include, for example, diluents, carriers, excipients, fillers, disintegrants, solubilizing agents, dispersing agents, preservatives, wetting agents, preservatives, stabilizers, buffering agents (e.g. phosphate, citrate, acetate, tartrate), suspending agents, emulsifiers, and penetration enhancing agents such as DMSO, as appropriate. The composition can also comprise suitable auxiliary substances, for example, solubilizing agents, dispersing agents, suspending agents and emulsifiers.

In certain embodiments, the composition further comprises suitable diluents, glidants, lubricants, acidulants, stabilizers, fillers, binders, plasticizers or release aids and other pharmaceutically acceptable excipients.

A complete description of pharmaceutically acceptable excipients can be found, for example, in Remington's Pharmaceutical Sciences (Mack Pub., Co., N.J. 1991) or other standard pharmaceutical science texts, such as the Handbook of Pharmaceutical Excipients (Shesky et al. eds., 8th ed. 2017).

In some embodiments, the composition for use in a disclosed method can be administered intra-gastrically, intravenously, intraperitoneally or intramuscularly, but other routes of administration are also possible.

Water may be used as a carrier and diluent in the composition. The use of other pharmaceutically acceptable solvents and diluents in addition to or instead of water is also acceptable.

Large macromolecules that are slowly metabolized, such as proteins, polysaccharides, polylactic acids, polyglycolic acids, polymeric amino acids, copolymers of amino acids, can also be used as carrier compounds for the composition. Pharmaceutically acceptable carriers in therapeutic compositions may additionally contain liquids, such as water, saline, glycerol or ethanol. Moreover, the said compositions may further comprise excipients, such as wetting agents or emulsifiers, buffering substances, and the like. Such excipients include, among others, diluents and carriers conventional in the art, and/or substances that promote penetration of the active compound into the cell, for example, DMSO, as well as preservatives and stabilizers.

The composition for use in a disclosed method may be presented in various dosage forms depending on the object of application; in particular, it may be formulated as a solution for injections.

The composition for use in a disclosed method may be administered systemically.

Suitable routes of administration include, for example, oral or parenteral administration, such as intravenous, intraperitoneal, intragastric as well as via drinking water. However, depending on a dosage form, the disclosed composition may be administered by other routes.

The disclosed composition can be co-administered with another appropriate agent or therapy.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes will occur to those skilled in the art. It is therefore to be understood that the appended claims are intended to cover all such modifications and changes as fall within the true spirit of the invention.

1. A method of treating an autoimmune disease in a patient in need thereof comprising administering a pharmaceutical composition comprising a therapeutically effective amount of an IL13 antagonist to said patient.

2. The method of claim 1, wherein the IL13 antagonist is an antagonist to IL13α1.

3. The method of claim 1, wherein the antagonist is an antibody.

4. The method of claim 1, wherein the autoimmune disease is a Th₂ T-cell mediated autoimmune disease.

5. The method of claim 1, wherein the autoimmune disease is Pemphigus vulgaris (PV).

6-11. (canceled)

12. A method of treating an autoimmune disease in a patient comprising administering a pharmaceutical composition comprising a therapeutically effective amount of an antagonist to a cytokine selected from the group consisting of IL-1α, IL-1β, IL-6, TNF-α, IL-8, and IL-21 to said patient.

13. The method of claim 12, wherein the antagonist is an antibody.

14. The method of claim 12, wherein the autoimmune disease is Pemphigus vulgaris (PV).

15. A method of treating a Pemphigus vulgaris (PV) patient by downregulating some of a panel of genes (protection signature genes) that are downregulated in healthy HLA-matched subjects of PV patients and/or by upregulating some of the protection signature genes that are upregulated in healthy HLA-matched subjects of PV patients.

16. The method of claim 15, wherein genes are downregulated or upregulated by use of siRNA, CRISPR, autoantibody blocking, molecular antagonists/agonists small molecule blocking/boosting.

17-18. (canceled)
